(19) United States
(12) Patent Application Publication
(10) Pub. No.: US 2026/0309675 A1
(43) Pub. Date: Oct. 8, 2026

(54) ULTRASONIC MEASUREMENT APPARATUS AND METHOD OF MEASURING FROZEN FISH

(71) Applicants: Fujitsu Limited, Kawasaki-shi (JP); Ishida Tec Co., Ltd., Yaizu-shi (JP)

(72) Inventors: Kenta YAMADA, Yaizu-shi (JP); Yoshinobu YAMAMURA, Yaizu-shi (JP); Hisashi ISHIDA, Yaizu-shi, (JP); Kanata SUZUKI, Kawasaki-shi (JP); Suguru YASUTOMI, Kawasaki-shi (JP); Akira SAKAI, Kawasaki-shi (JP)

(73) Assignees: Fujitsu Limited, Kawasaki-shi (JP); Ishida Tec Co., Ltd., Yaizu-shi (JP)

(21) Appl. No.: 19/568,851

(22) Filed: Mar. 17, 2026

(30) Foreign Application Priority Data
Apr. 8, 2025 (JP) 2025-063868

(51) Int. Cl. G01N 29/04; B65G 43/08; G01N 33/12

(52) U.S. Cl. CPC G01N 29/04; B65G 43/08; B65G 2201/0202; B65G 2203/042; G01N 33/12

(57) ABSTRACT

An ultrasonic measurement apparatus includes a conveyor that conveys a frozen fish from one side to another side, a first measurer provided on the another side of the conveyor, the first measurer contacting the frozen fish conveyed by the conveyor to perform ultrasonic measurement on the frozen fish, a support member that supports the frozen fish contacted by the first measurer, and a heater provided in the first measurement, the heater increasing a surface temperature of the frozen fish.

### CROSS-REFERENCE TO RELATED APPLICATION

This application is based upon and claims the benefit of priority of Japanese Patent Application No. 2025-063868 filed on Apr. 8, 2025, the entire contents of which are incorporated herein by reference.

### FIELD

A certain aspect of the present embodiments relates to an ultrasonic measurement apparatus and a method of measuring a frozen fish.

### BACKGROUND

Conventionally, an object has been inspected using ultrasonic waves (for example, see Patent Document 1: Japanese Patent Application Publication No. 2022-153451). When an inspection using ultrasonic waves is performed, a couplant is supplied between an inspection target and a probe. For example, an ultrasonic inspection device in Patent Document 1 includes a self-lubricating material as a coupling element that comes into contact with an object and is acoustically coupled to the object. In addition, an aspect in which the self-lubricating material contains a hydrophilic elastomer is disclosed.

### SUMMARY

According to an aspect of the present disclosure, there is provided an ultrasonic measurement apparatus including: a conveyor that conveys a frozen fish from one side to another side; a first measurer provided on the another side of the conveyor, the first measurer contacting the frozen fish conveyed by the conveyor to perform ultrasonic measurement on the frozen fish; a support member that supports the frozen fish contacted by the first measurer; and a heater provided in the first measurement, the heater increasing a surface temperature of the frozen fish.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF THE DRAWINGS

FIG. 1 is a perspective view illustrating an example of an ultrasonic measurement apparatus according to an embodiment.

FIG. 2 is a front view illustrating an example of the ultrasonic measurement apparatus according to the embodiment.

FIG. 3 is a plan view illustrating an example of the ultrasonic measurement apparatus according to the embodiment.

FIG. 4A is a right side view illustrating an example of the ultrasonic measurement apparatus according to the embodiment.

FIG. 4B is a left side view illustrating an example of the ultrasonic measurement apparatus according to the embodiment.

FIG. 5 is a functional block diagram illustrating an example of a controller included in the ultrasonic measurement apparatus according to the embodiment.

FIG. 6A is a front view of a conveyance unit included in the ultrasonic measurement apparatus according to the embodiment.

FIG. 6B is a plan view of the conveyance unit included in the ultrasonic measurement apparatus according to the embodiment.

FIG. 7A is a cross-sectional view of the conveyance unit according to the embodiment taken along a line B-B in FIG. 6B.

FIG. 7B is a cross-sectional view of the conveyance unit according to the embodiment taken along a line A-A in FIG. 6A.

FIG. 7C is a cross-sectional view of the conveyance unit according to the embodiment taken along a line C-C in FIG. 6A.

FIG. 8A is a plan view of a second support portion and a first measurement unit included in the ultrasonic measurement apparatus according to the embodiment.

FIG. 8B is a right side view of the second support unit and the first measurement unit included in the ultrasonic measurement apparatus according to the embodiment.

FIG. 9 is a cross-sectional view of the second support unit and the first measurement unit included in the ultrasonic measurement device according to the embodiment, taken along a line D-D in FIG. 8B.

FIG. 10 is a perspective view of the second support unit included in the ultrasonic measurement apparatus according to the embodiment.

FIG. 11 is a plan view illustrating one of a pair of pressing units included in the second support unit included in the ultrasonic measurement apparatus according to the embodiment.

FIG. 12A is a cross-sectional view of one of the pair of pressing units included in the second support unit included in the ultrasonic measurement apparatus according to the embodiment, taken along a line E-E in FIG. 11.

FIG. 12B is a cross-sectional view of one of the pair of pressing units included in the second support unit included in the ultrasonic measurement apparatus according to the embodiment, taken along a line F-F in FIG. 11.

FIG. 13A is a front view of a probe unit included in the ultrasonic measurement apparatus according to the embodiment.

FIG. 13B is a cross-sectional view of the probe unit included in the ultrasonic measurement apparatus according to the embodiment, taken along a line G-G in FIG. 13A.

FIG. 13C is a side view of the probe unit included in the ultrasonic measurement apparatus according to the embodiment.

FIG. 14 is a schematic view illustrating a schematic configuration of a measurement head.

FIG. 15 is a schematic view illustrating a schematic configuration of a defrosting brush.

### DESCRIPTION OF EMBODIMENTS

Measurement using ultrasonic waves is used in various fields, and the use of ultrasonic measurement of measuring the quality of frozen fish is also being studied. Conventionally, the quality of frozen fish has been determined by, for example, observing the cross-section of the tail of a cut piece of frozen fish. Such quality determination based on the observation of the cross section requires the skill of a determiner. It is expected that the quality of the frozen fish can be easily determined by utilizing the measurement using ultrasonic waves.

However, the surface of the frozen fish is covered with frost. Frost contains a lot of air bubbles, and therefore is less likely to transmit sound. Thus, frost reduces the accuracy of ultrasonic measurement. Patent Document 1 does not assume that frozen fish is targeted, and no measures against frost are considered.

In one aspect, the present disclosure aims to improve the accuracy of ultrasonic measurement on frozen fish.

Hereinafter, an embodiment of the present disclosure will be described with reference to the accompanying drawings. However, in the drawings, the dimensions, ratios, and the like of the respective parts may not be illustrated so as to completely match the actual ones. The drawings may be on different scales from one drawing to another. In addition, depending on the drawings, for convenience of description, there are cases where constituent elements that are actually present are omitted, or the dimensions are exaggerated compared to the actual dimensions.

### Embodiment

### [Configuration of Ultrasonic Measurement Apparatus]

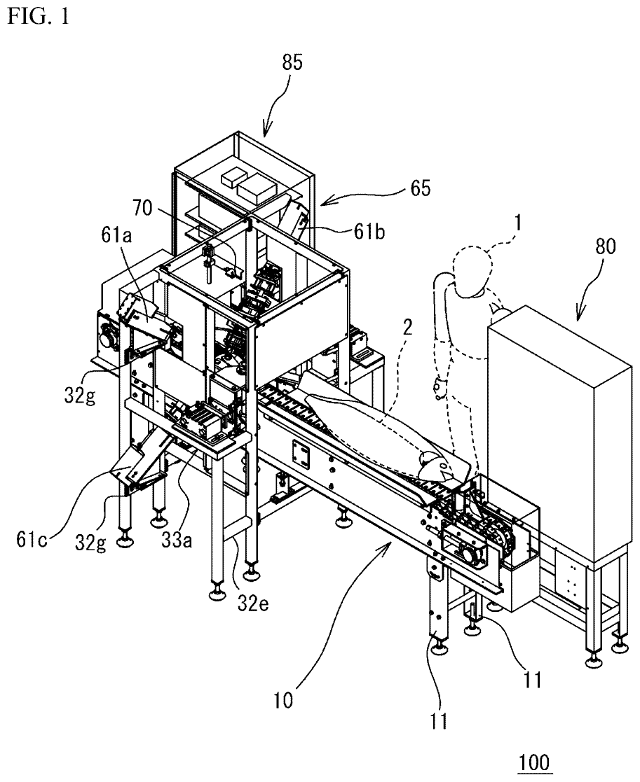

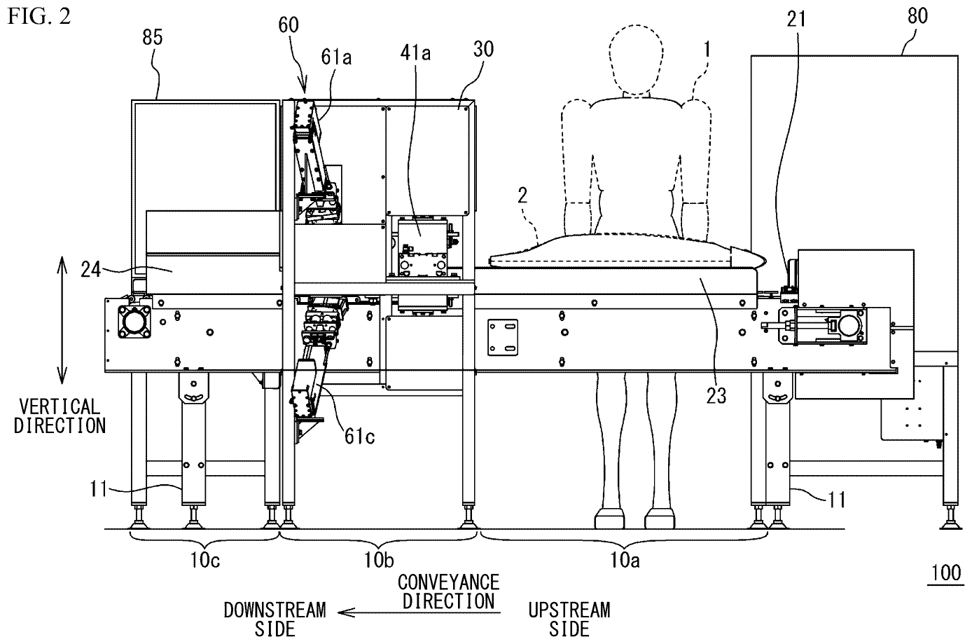

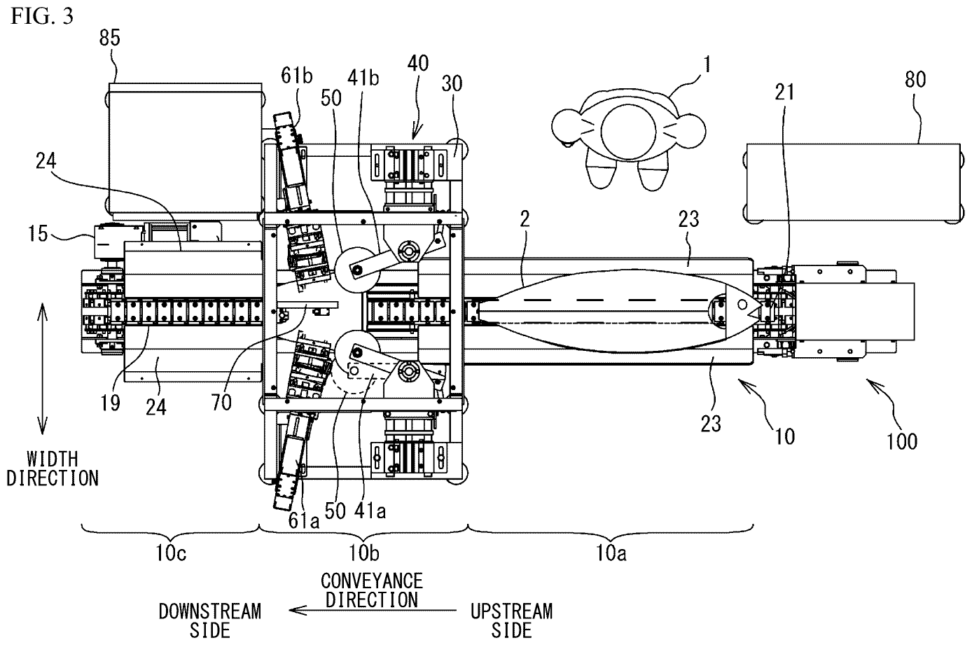

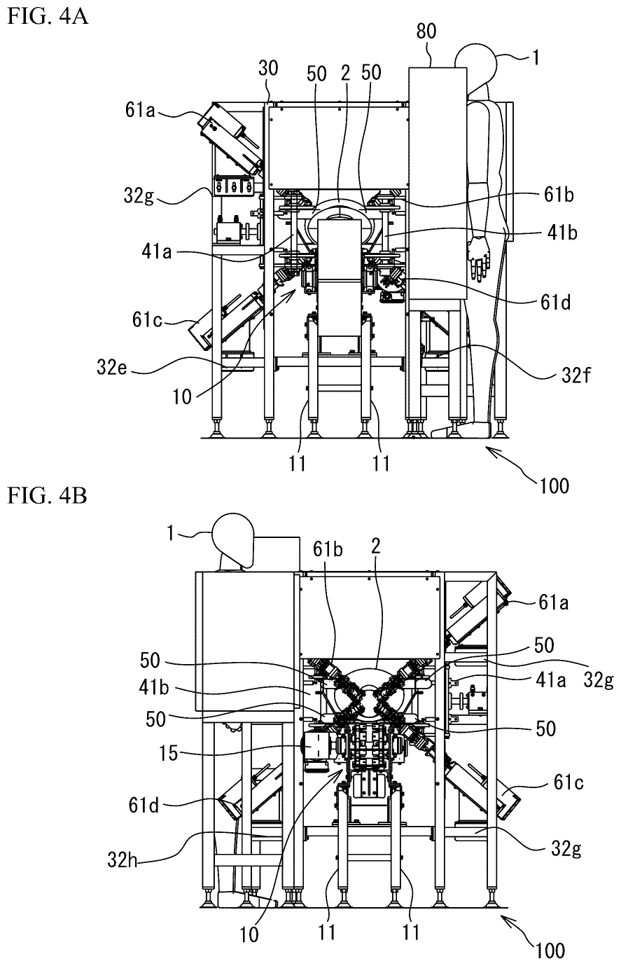

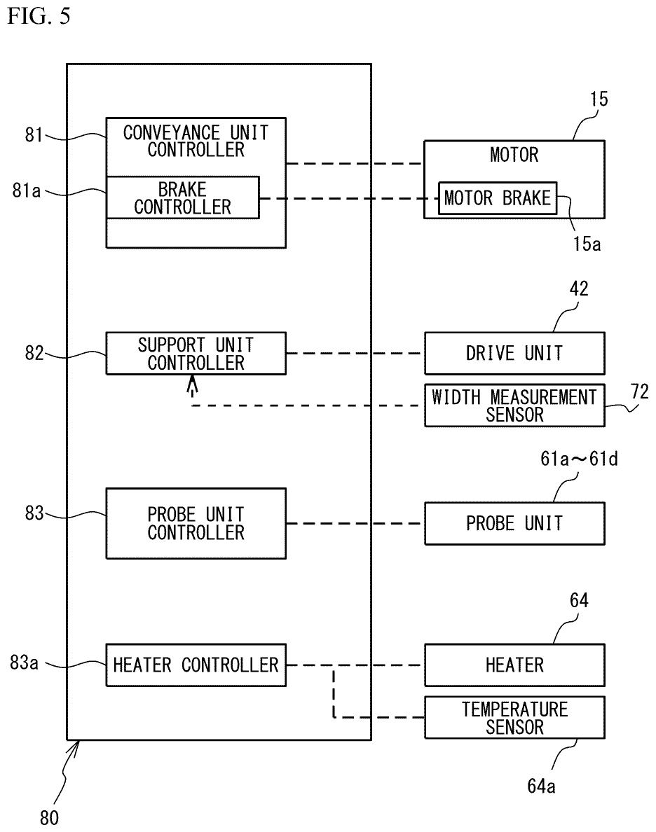

First, a schematic configuration of an ultrasonic measurement apparatus 100 according to an embodiment will be described with reference to FIGS. 1 to 5. FIG. 1 is a perspective view illustrating an example of the ultrasonic measurement apparatus 100 according to the embodiment. FIG. 2 is a front view illustrating the example of the ultrasonic measurement apparatus 100 according to the embodiment. FIG. 3 is a plan view illustrating the example of the ultrasonic measurement apparatus according to the embodiment. FIG. 4A is a right side view illustrating the example of the ultrasonic measurement apparatus according to the embodiment. FIG. 4B is a left side view illustrating the example of the ultrasonic measurement apparatus according to the embodiment. FIG. 5 is a functional block diagram illustrating an example of a controller included in the ultrasonic measurement apparatus according to the embodiment.

The ultrasonic measurement apparatus 100 includes a conveyance unit 10 including a work pusher 21 that functions as a first support unit, a second support unit 40, and a first measurement unit 60. The ultrasonic measurement apparatus 100 includes a width measurement unit 70, a controller 80, and an information processing unit 85. In FIGS. 2 and 3, the conveyance unit 10 conveys a frozen fish 2 along the conveyance direction illustrated in the figures. In the examples illustrated in FIGS. 2 and 3, the conveyance direction is from the right side to the left side in the figures. Therefore, the right side in the figures is an upstream side (one side) in the conveyance direction by the conveyance unit 10, and the left side in the figure is a downstream side (the other side) in the conveyance direction by the conveyance unit 10. In the conveyance unit 10, a loading region 10a, a measurement region 10b, and a discharge region 10c are set in order from the upstream side to the downstream side. The second support unit 40, the first measurement unit 60, and the width measurement unit 70 are provided in the measurement region 10b. The frozen fish 2 is loaded into the conveyance unit 10 by an operator 1 in the loading region 10a, for example, and is conveyed toward the downstream side by the conveyance unit 10. In the following description, a direction intersecting the conveyance direction is referred to as a width direction (intersecting direction) (see FIG. 3), and a direction different from the conveyance direction and the width direction is referred to as a vertical direction (see FIG. 2). In the present embodiment, the conveyance direction and the width direction are orthogonal to each other. The vertical direction is orthogonal to the conveyance direction and the width direction. The width direction is a short-side direction of the frozen fish 2.

The second support unit 40 restrains the frozen fish 2 so that the frozen fish 2 being inspected does not move in the vertical direction and the width direction. The first measurement unit 60 is in contact with the surface of the frozen fish 2 to be measured and performs measurement by ultrasonic waves.

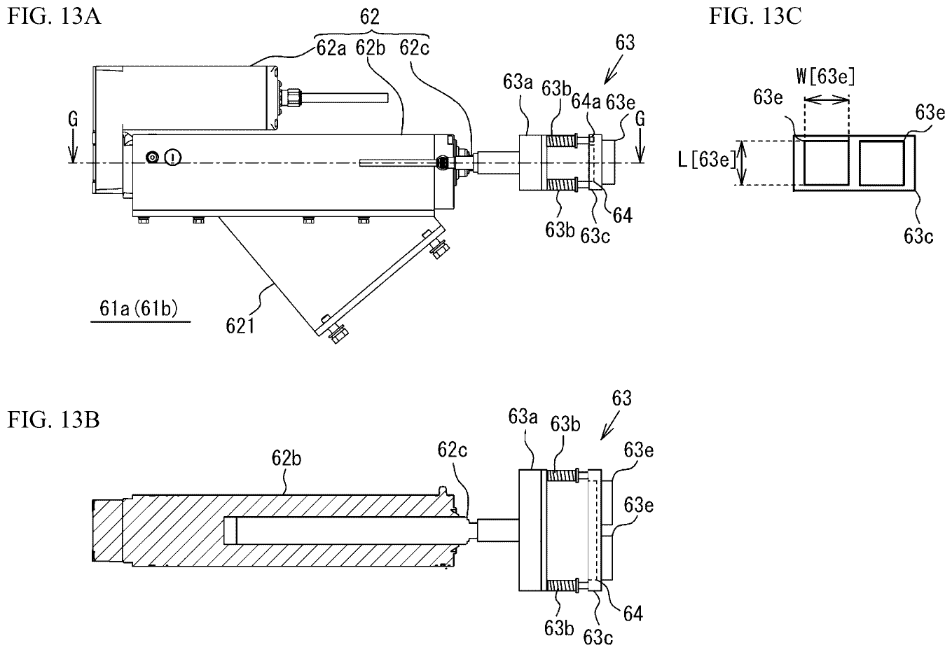

The controller 80 includes a conveyance unit controller 81, a brake controller 81a, a support unit controller 82, a probe unit controller 83, and a heater controller 83a. The conveyance unit controller 81 controls the conveyance unit 10, specifically, controls a drive motor 15. The details of the conveyance unit 10 will be described later. The brake controller 81a controls a motor brake 15a provided in the drive motor 15 included in the conveyance unit 10. The drive motor 15 will be described later. The support unit controller 82 controls a drive unit 42 of the second support unit 40. The second support unit 40 will be described in detail later. The probe unit controller 83 controls the operations of a first probe unit 61a, a second probe unit 61b, a third probe unit 61c, and a fourth probe unit 61d (see FIGS. 4A and 4B, etc.) included in the first measurement unit 60. These probe units will be described later. The heater controller 83a controls a heater 64 (see FIGS. 13A and 13B) included in each probe unit. The heater 64 will be described later.

The information processing unit 85 includes a central processing unit (CPU), a read-only memory (ROM), a random access memory (RAM), a storage (such as an HDD or an SSD), a network interface, a portable storage medium drive, and the like, which are not illustrated. The information processing unit 85 determines the quality of the frozen fish 2 based on the measurement values acquired by the probe units 61a to 61d. The quality of the frozen fish 2 is determined by comparing the measured values with data in which a large number of measured values are associated with the quality of the frozen fish 2.

### [Configuration of Conveyance Unit]

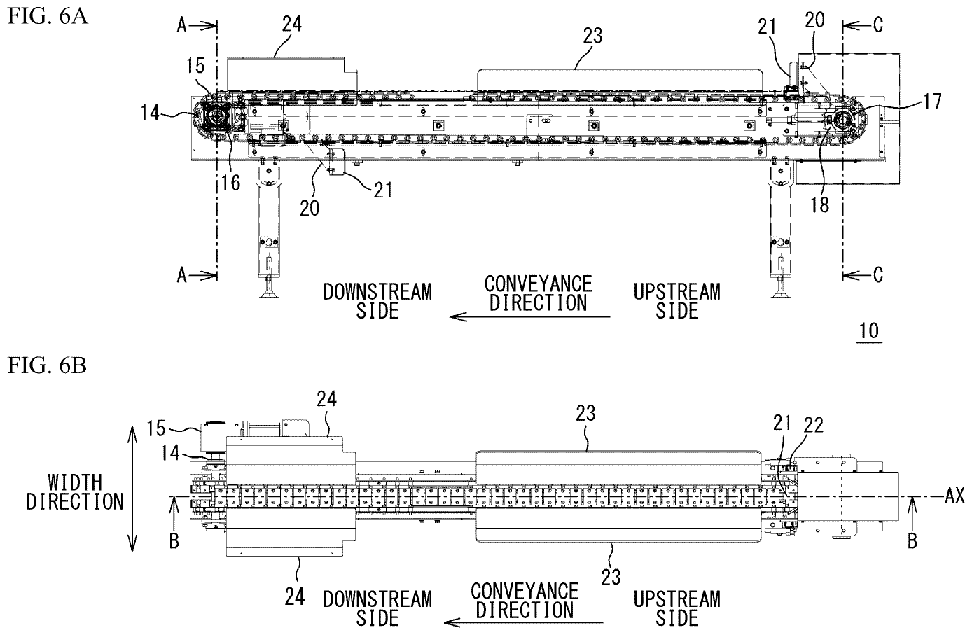

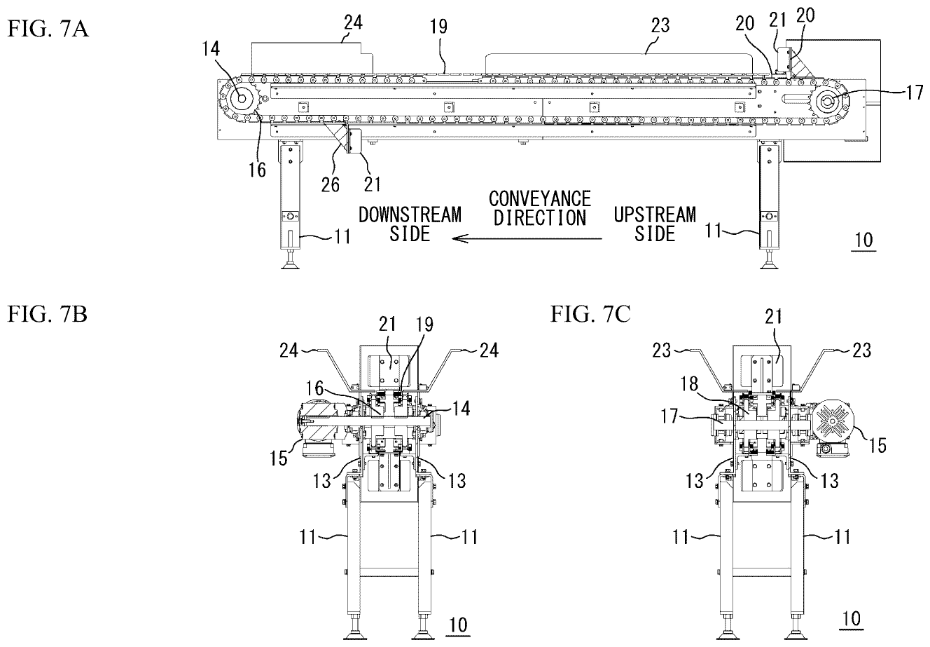

Next, the conveyance unit 10 will be described with reference to FIGS. 6A to 7C. FIG. 6A is a front view of the conveyance unit 10 included in the ultrasonic measurement apparatus 100 according to the embodiment. FIG. 6B is a plan view of the conveyance unit 10 included in the ultrasonic measurement apparatus 100 according to the embodiment. FIG. 7A is a cross-sectional view of the conveyance unit 10 according to the embodiment, taken along a line B-B in FIG. 6B. FIG. 7B is a cross-sectional view of the conveyance unit 10 according to the embodiment, taken along a line A-A in FIG. 6A. FIG. 7C is a cross-sectional view of the conveyance unit 10 according to the embodiment, taken along a line C-C in FIG. 6A. The line B-B coincides with a center line AX along the conveyance direction of the conveyance chain 19 included in the conveyance unit 10.

The conveyance unit 10 includes four leg portions 11. An adjuster bolt is provided at the lower end of each leg portion 11, and the height of the leg portion 11 can be adjusted individually. Referring to FIGS. 7A and 7B, conveyor frames 13 are mounted on the leg portions 11. The conveyor frames 13 are formed by a pair of two members disposed to face each other in a direction orthogonal to the conveyance direction. The conveyor frame 13 extends along the conveyance direction.

A drive shaft 14 is rotatably supported on the downstream side of the conveyor frame 13. A driven shaft 17 is rotatably provided on the upstream side of the conveyor frame 13.

The drive motor 15 is connected to the drive shaft 14. The drive motor 15 has the motor brake 15a (see FIG. 5) incorporated therein. The motor brake 15a corresponds to a brake device. Since the structure of the motor brake 15a is conventionally known, a detailed description thereof will be omitted here. The drive shaft 14 is provided with a driving sprocket 16. The driven shaft 17 is provided with a driven sprocket 18. The drive-side mechanism and the driven-side mechanism may be switched between the upstream side and the downstream side.

A conveyance chain 19 is wound around the driving sprocket 16 and the driven sprocket 18. The conveyance chain 19 is an endless chain, and a chain pusher 20 and the work pusher 21 are provided. The chain pusher 20 is a support component of the work pusher 21. The work pusher 21 has a function of conveying the frozen fish 2 placed on the conveyance chain 19 and conveyed, while pushing the frozen fish 2 downstream, integrally with the chain pusher 20 when the conveyance unit 10 is operated. The work pusher 21 corresponds to the first support unit as described above, and also functions as a stopper member that prevents the frozen fish 2 from moving mainly toward the upstream side of the conveyance unit 10 when the probe units 61a to 61d are pressed against the frozen fish 2 during measurements. The stopper member may be provided separately from the conveyance chain 19.

The conveyance unit 10 includes a photoelectric sensor 22 near the upstream side of the conveyance chain 19 (see FIG. 6B). The photoelectric sensor 22 is a loading standby position stop command sensor for the work pusher 21 and the chain pusher 20. The photoelectric sensor 22 detects the work pusher 21 and the chain pusher 20 moving with the driving of the conveyance chain 19, and transmits a conveyance stop signal for stopping the work pusher 21 and the chain pusher 20 at a predetermined position.

The conveyance unit 10 includes a first work guide 23 and a second work guide 24. The first work guide 23 is provided on the upstream side of the conveyance unit 10. The second work guide 24 is provided on the downstream side of the conveyance unit 10. As illustrated in FIG. 6B, each of the first work guide 23 and the second work guide 24 is formed by a pair of components facing each other in the width direction. The first work guide 23 and the second work guide 24 guide the frozen fish 2 so that the frozen fish 2 stays on the conveyance chain 19 and is conveyed from the upstream side to the downstream side of the conveyance unit 10. The first work guide 23 and the second work guide 24 are provided with a space therebetween along the conveyance direction. The probe units 61a to 61d are disposed between the first work guide 23 and the second work guide 24.

In the present embodiment, the conveyance chain 19 is supported so as to be operable by the leg portions 11 and the conveyor frames 13, but the supporting mode of the conveyance chain 19 is not limited thereto. Any mode that can support the conveyance chain 19 so as to be operable can be adopted.

### [Configuration of Measurement Unit Frame]

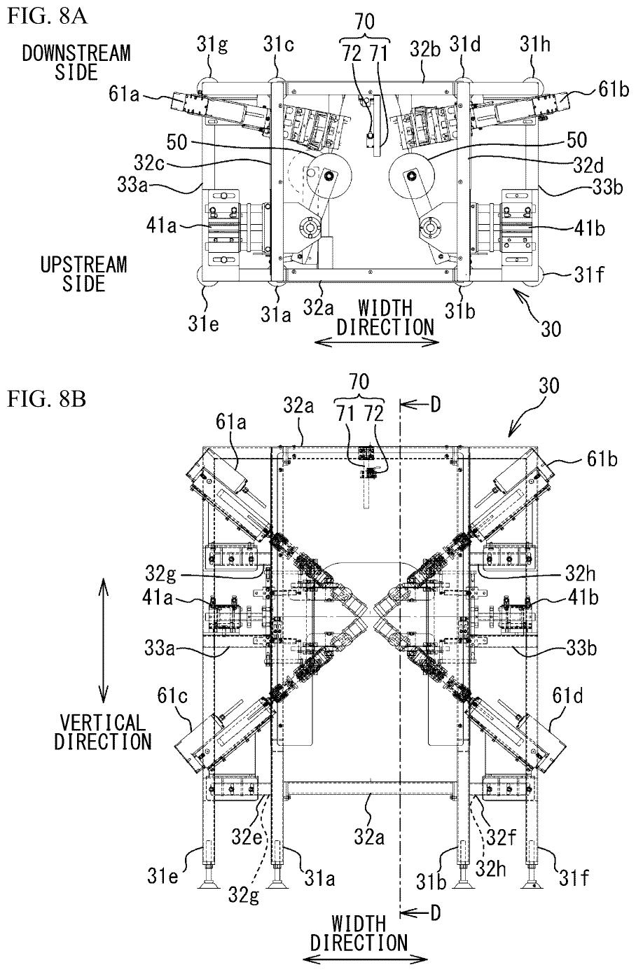

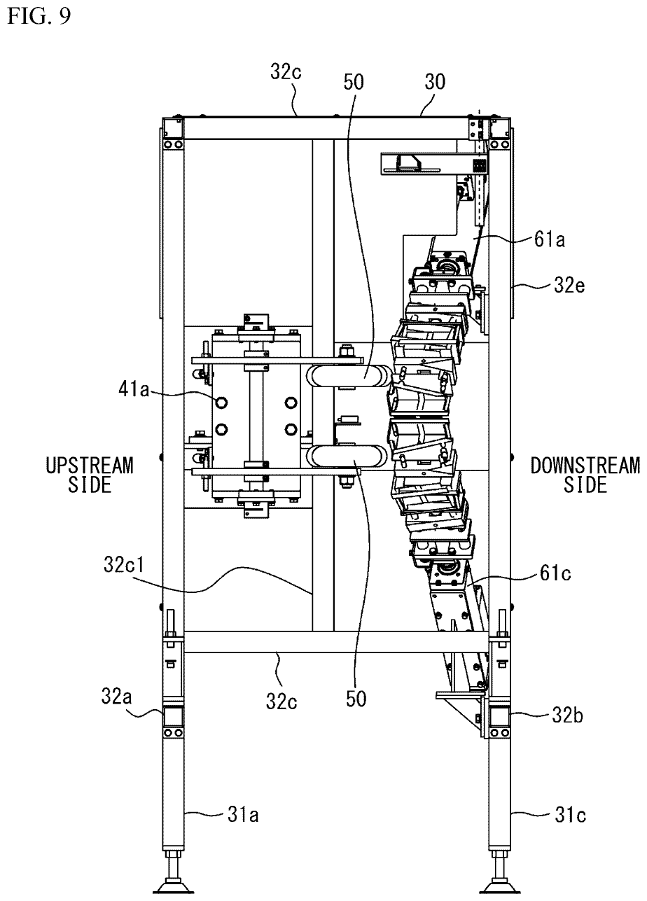

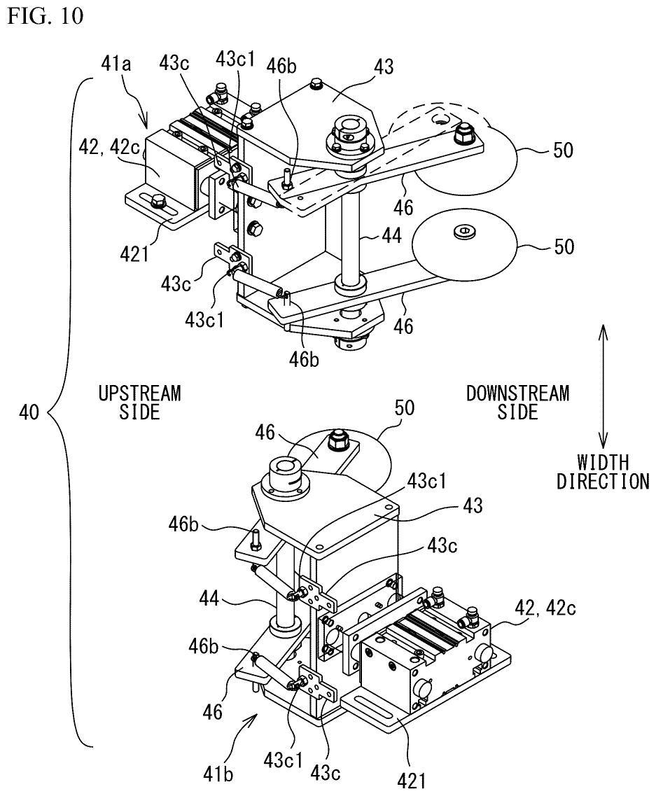

Referring to FIG. 10, the second support unit 40 includes a first pressing unit 41a and a second pressing unit 41b disposed to face each other in the width direction. Referring to FIGS. 8A to 9, the first pressing unit 41a and the second pressing unit 41b are mounted on a measurement unit frame 30 together with the probe units 61a to 61d. First, the measurement unit frame 30 will be described with reference to FIGS. 1, 8A to 9, and the like. FIG. 8A is a plan view of the second support unit 40 and the first measurement unit 60 included in the ultrasonic measurement apparatus 100 according to the embodiment. FIG. 8B is a right side view of the second support unit 40 and the first measurement unit 60 included in the ultrasonic measurement apparatus 100 according to the embodiment. FIG. 9 is a cross-sectional view of the second support unit 40 and the first measurement unit 60 included in the ultrasonic measurement apparatus 100 according to the embodiment, taken along a line D-D in FIG. 8B.

The measurement unit frame 30 includes a first leg portion 31a to an eighth leg portion 31h. The first leg portion 31a is disposed on the upstream side of the measurement unit frame 30 and on the inside in the width direction of the measurement unit frame 30. A second leg portion 31b is disposed adjacent to the first leg portion 31a in the width direction, on the upstream side of the measurement unit frame 30 and on the inside in the width direction of the measurement unit frame 30. A third leg portion 31c is disposed on the downstream side of the measurement unit frame 30 and on the inside in the width direction of the measurement unit frame 30. A fourth leg portion 31d is disposed adjacent to the third leg portion 31a in the width direction, on the downstream side of the measurement unit frame 30 and on the inside in the width direction of the measurement unit frame 30. A fifth leg portion 31e is disposed on the upstream side of the measurement unit frame 30 and on the outside in the width direction of the first leg portion 31a. A sixth leg portion 31f is disposed on the upstream side of the measurement unit frame 30 and on the outside in the width direction of the second leg portion 31b. A seventh leg portion 31g is disposed on the downstream side of the measurement unit frame 30 and on the outside in the width direction of the third leg portion 31c. The eighth leg portion 31h is disposed on the downstream side of the measurement unit frame 30 and on the outside in the width direction of the fourth leg portion 31d. The fifth leg portion 31e and the sixth leg portion 31f are shorter than the other leg portions, and the upper ends of the fifth leg portion 31e and the sixth leg portion 31f are located lower than the upper ends of the other leg portions.

The measurement unit frame 30 includes a first coupling member 32a to an eighth coupling member 32h. The first coupling member 32a couples the first leg portion 31a and the second leg portion 31b. The first coupling member 32a is provided one each on the upper and lower sides. A second coupling member 32b couples the third leg portion 31c and the fourth leg portion 31d. The second coupling member 32b is provided one each on the upper and lower sides. A third coupling member 32c couples the first leg portion 31a and the third leg portion 31c. The third coupling member 32c is provided one each on the upper and lower sides. The third coupling members 32c on the upper and lower sides are connected by a first reinforcing member 32c1 extending in the vertical direction. The position of the third coupling member 32c on the lower side in the vertical direction is higher than the height positions of the first coupling member 32a and the second coupling member 32b. A fourth coupling member 32d couples the second leg portion 31b and the fourth leg portion 31d. The fourth coupling member 32d is provided one each on the upper and lower sides. The fourth coupling members 32d on the upper and lower sides are connected by a second reinforcing member (not illustrated) extending in the vertical direction. The position of the fourth coupling member 32d on the lower side in the vertical direction is higher than the height positions of the first coupling member 32a and the second coupling member 32b, and is substantially the same position as the third coupling member 32c on the lower side. A fifth coupling member 32e couples the first leg portion 31a and the fifth leg portion 31e. Only one fifth coupling member 32e is provided. A sixth coupling member 32f couples the second leg portion 31b and the sixth leg portion 31f. Only one sixth coupling member 32f is provided. A seventh coupling member 32g couples the third leg portion 31c and the seventh leg portion 31g. The seventh coupling member 32g is provided one each on the upper and lower sides (see FIGS. 1 and 8B). The first probe unit 61a is mounted on the seventh coupling member 32g on the upper side (see FIG. 8B). The third probe unit 61c is mounted on the seventh coupling member 32g on the lower side (see FIG. 8B). The eighth coupling member 32h couples the fourth leg portion 31d and the eighth leg portion 31h. The eighth coupling member 32h is provided one each on the upper and lower sides (see FIG. 8B). The second probe unit 61b is mounted on the eighth coupling member 32h on the upper side (see FIG. 8B). The fourth probe unit 61d is mounted on the eighth coupling member 32h on the lower side (see FIG. 8B).

The measurement unit frame 30 includes a first mounting bracket 33a and a second mounting bracket 33b. The first mounting bracket 33a is an L-shaped member and connects the first leg portion 31a, the fifth leg portion 31e, and the seventh leg portion 31g. The first mounting bracket 33a is provided such that an L-shaped corner portion is supported by an upper end portion of the fifth leg portion 31e. The first pressing unit 41a is mounted on the first mounting bracket 33a. The second mounting bracket 33b is an L-shaped member similar to the first mounting bracket 33a, and connects the second leg portion 31b, the sixth leg portion 31f, and the eighth leg portion 31h. The second mounting bracket 33b is provided such that an L-shaped corner portion is supported by an upper end portion of the sixth leg portion 31f. The second pressing unit 41b is mounted on the second mounting bracket 33b.

The measurement unit frame 30 of the present embodiment is an example of a structure that supports the first pressing unit 41a, the second pressing unit 41b, and the probe units 61a to 61d. The structure for supporting the first pressing unit 41a, the second pressing unit 41b, and the probe units 61a to 61d is not limited thereto. The structure for supporting the first pressing unit 41a, the second pressing unit 41b, and the probe units 61a to 61d can be changed as appropriate.

### [Configuration of Second Support Unit]

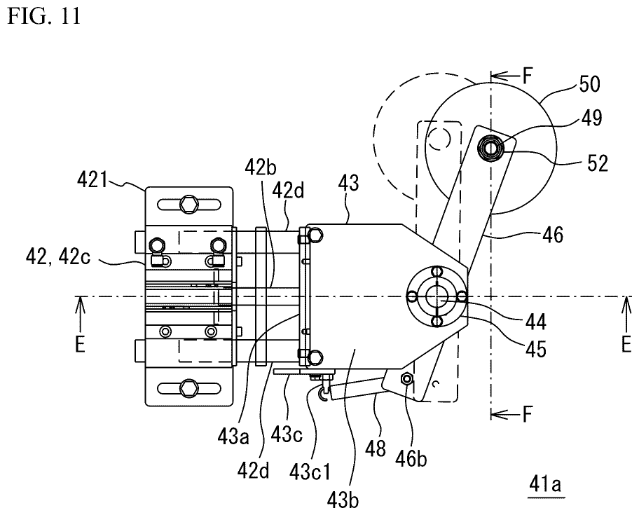

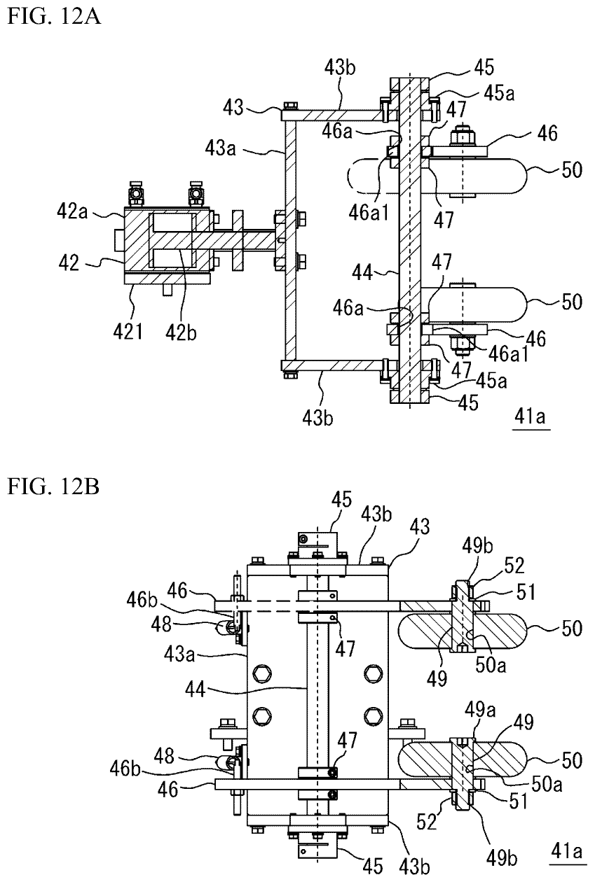

Next, the second support unit 40 will be described with reference to FIGS. 8A to 12B. FIG. 10 is a perspective view of the second support unit 40 included in the ultrasonic measurement apparatus 100 according to the embodiment. FIG. 11 is a plan view illustrating one pressing unit 41a of the pair of pressing units 41b and 41a included in the second support unit 40 included in the ultrasonic measurement apparatus 100 according to the embodiment. FIG. 12A is a cross-sectional view of one pressing unit 41a of the pair of pressing units 41b and 41a included in the second support unit 40 included in the ultrasonic measurement apparatus 100 according to the embodiment, taken along a line E-E in FIG. 11. FIG. 12B is a cross-sectional view of one pressing unit 41a of the pair of pressing units 41b and 41a included in the second support unit 40 included in the ultrasonic measurement apparatus 100 according to the embodiment, taken along a line F-F in FIG. 11.

As described above, the second support unit 40 includes the first pressing unit 41a and the second pressing unit 41b (see FIG. 10). The first pressing unit 41a and the second pressing unit 41b are disposed to face each other in the width direction. The first pressing potion 41a and the second pressing unit 41b are provided so as to be line-symmetrical with respect to the center line AX of the conveyance chain 19 along the conveying direction of the frozen fish 2. Therefore, the first pressing unit 41a and the second pressing unit 41b have the same components, although the components are arranged line-symmetrically with each other. Therefore, the same reference numerals are given to the common components. In the following description, the first pressing unit 41a will be described as a representative.

The first pressing unit 41a includes the drive unit 42. The drive unit 42 of the present embodiment is an air cylinder driven by compressed air. However, the drive unit 42 may be an electric actuator or another actuator.

The drive unit 42 includes a drive cylinder unit 42a, a drive piston 42b, a guide unit 42c, and a guide piston 42d. Compressed air is supplied from an air source (not illustrated) to the drive cylinder unit 42a. The drive piston 42b moves to protrude and retract with respect to the drive cylinder unit 42a in accordance with the state of introduction and discharge of compressed air with respect to the drive cylinder unit 42a. The guide piston 42d is provided slidably with respect to the guide unit 42c. The guide piston 42d is provided in parallel with the drive piston 42b. By providing the guide piston 42d, the drive piston 42b is suppressed from being inclined and is stably driven.

The drive unit 42 includes a cylinder bracket 421. The drive unit 42 of the first pressing unit 41a is mounted on the first mounting bracket 33a via the cylinder bracket 421. The drive unit 42 of the second pressing unit 41b is mounted on the second mounting bracket 33b via the cylinder bracket 421.

The first pressing unit 41a includes a base unit 43. The base unit 43 includes a back plate 43a and a pair of shaft support plates 43b provided to face each other in the vertical direction. One of the pair of shaft support plates 43b is connected to an upper end portion of the back plate 43a, and the other of the pair of shaft support plates 43b is connected to a lower end portion of the back plate 43a. Thus, the base unit 43 has a U-shape. The base unit 43 is provided with spring post bases 43c. The spring post base 43c is provided with a first spring post 43c1.

The distal end portion of the drive piston 42b and the distal end portion of the guide piston 42d are mounted on the back plate 43a of the base unit 43. Accordingly, the base unit 43 can be moved to protrude and retract with respect to the drive cylinder unit 42a by the operation of the drive unit 42. That is, the base unit 43 can approach and separate from the frozen fish 2 conveyed by the conveyance unit 10.

A shaft 44 is rotatably provided on the base unit 43. The shaft 44 is rotatably supported by shaft holders 45 provided at the upper and lower shaft support plates 43b. The shaft holder 45 is fixed to the respective shaft support plates 43b by bolts 45a.

The shaft 44 is provided with plate-shaped arm members 46. The shaft 44 is provided with the two arm members 46. The two arm members 46 are provided in parallel with a space therebetween along the axial direction of the shaft 44. Each arm member 46 includes an insertion hole 46a through which the shaft 44 is inserted. An annular flange bushing 46a1 is fitted into the insertion hole 46a. The shaft 44 is fitted into the inner peripheral side of the flange bushing 46a1. The shaft 44 is provided with collar members 47 that are respectively disposed on the upper and lower surfaces of the arm members 46 and interpose the arm members 46 from the vertical directions (i.e., upper and lower directions). Thus, the arm members 46 can rotate integrally with the shaft 44. A second spring post 46b is provided on one end side of the arm member 46.

A spring member 48 is stretched between the first spring post 43c1 and the second spring post 46b. When the arm member 46 rotates counterclockwise about the shaft 44 in the plan view illustrated in FIG. 11, the spring member 48 is pulled and exerts a pulling force that rotates the arm member 46 clockwise.

A roller member 50 is mounted on the other end portion of the arm member 46 different from the one end portion where the second spring post 46b is provided. The roller member 50 is rotatably provided by a pin member 49. The pin member 49 includes a head portion 49a on one end side and a screw portion 49b on the other end side. The pin member 49 is inserted into an insertion hole 50a provided in the roller member 50, and a washer 51 and a nut 52 cover the screw portion 49b. Thus, the roller member 50 is supported on the pin member 49 with play.

The roller members 50 are provided on the first pressing unit 41a with a gap in the vertical direction. The roller member 50 is also provided on the second pressing unit 41b disposed to face the first pressing unit 41a. That is, the second support unit 40 includes four roller members 50. The four roller members 50 are arranged so as to surround the frozen fish 2 conveyed by the conveyance unit 10. Specifically, the four roller members 50 are arranged so as to surround the frozen fish 2 from the vertical direction and the width direction thereof. Such an arrangement of the four roller members 50 is realized by mounting the first pressing unit 41a on the first mounting bracket 33a and mounting the second pressing unit 41b on the second mounting bracket 33b.

### [Configuration of First Measurement Unit]

Next, the first measurement unit 60 (referring to FIG. 2) will be described. The first measurement unit 60 includes the first probe unit 61a, the second probe unit 61b, the third probe unit 61c, and the fourth probe unit 61d. That is, the first measurement unit 60 includes four probe units. However, the number of probe units is not limited thereto. By providing a plurality of probe units, the accuracy of the measurement inspection can be improved. Further, even when some of the plurality of probe units are in a malfunction state, the possibility of obtaining a measurement result increases.

The probe units 61a to 61d have a common structure, but the arrangement and the orientation of the components are different depending on the positions where the probe units are arranged. Therefore, in the following description, the first probe unit 61a will be described as a representative. The arrangement and the orientation of the components of the first probe unit 61a are the same as those of the components of the second probe unit 61b. The arrangement and the orientation of the components of the third probe unit 61c are the same as those of the components of the fourth probe unit 61d.

The first probe unit 61a includes an electric actuator 62. The electric actuator 62 functions as a pressing unit. Each of the electric actuators 62 includes a motor 62a, a cylinder 62b, and a sliding rod 62c. The sliding rod 62c is provided slidably with respect to the cylinder 62b. The sliding rod 62c is moved along the axial direction via a ball screw (not illustrated) driven by the motor 62a. Since the electric actuators 62 are conventionally known, a detailed description of a mechanism for transmitting the rotation of the motor 62a to the sliding rod 62c via the ball screw will be omitted. Further, another actuator may be used instead of the electric actuator 62.

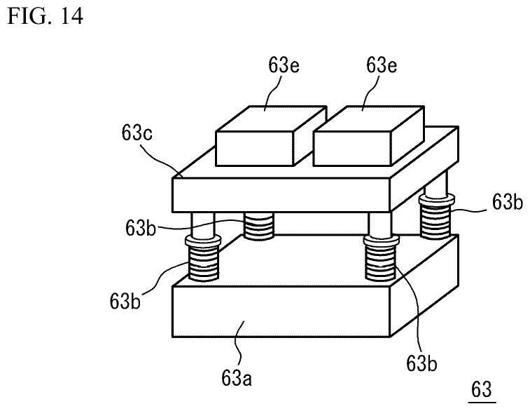

A measurement head 63 is provided at the distal end of the sliding rod 62c (see FIGS. 13A to 14). The measurement head 63 forms a probe. The measurement head 63 includes a base plate 63a, support springs 63b, and a pad holding plate 63c. The base plate 63a is connected to the sliding rod 62c. The support springs 63b are disposed between the base plate 63a and the pad holding plate 63c. In the present embodiment, four support springs 63b are provided. The support springs 63b allow the pad holding plate 63c to change its posture in accordance with the shape of the frozen fish 2. The number of support springs 63b is not limited to any particular number and size as long as the pad holding plate 63c can change its posture appropriately. In order to change the posture of the pad holding plate 63c, for example, a swing mechanism may be incorporated.

The pad holding plate 63c is provided with a pair of pads 63e. One of the pair of pads 63e is a transmission pad, and the other thereof is a reception pad. The shape of the contact surface of each pad 63e with the frozen fish 2 is a rectangle of L [63e]×W [63e]. The dimensions L [63e] and W [63e] can be set as appropriate. The shape is not limited to a rectangle, and may be another shape. The pad 63e is made of silicon rubber. Since the silicone rubber has flexibility, it can be deformed in accordance with the shape of the surface of the frozen fish 2, and the adhesion to the surface of the frozen fish 2 can be enhanced. Note that, instead of the pad made of silicone rubber, another pad using another material having flexibility may be used.

The pad holding plate 63c is provided with the heater 64 functioning as a heating unit for increasing the temperature of the pad 63e and increasing the surface temperature of the frozen fish 2, and a temperature sensor 64a. The heater 64 increases the temperature of the measurement head 63. The temperature sensor 64a measures the temperature of the measurement head 63. A heater controller 93a (see FIG. 5) can adjust the output of the heater 64 based on the measurement value of the temperature sensors 64a. The inclusion of the heater 64 can suppress a change in the property of the pad 63e due to a low temperature. It is considered that the pad 63e is easily deformed in accordance with the surface shape of the frozen fish 2 by using a material having a low hardness, and easily comes into close contact with the surface of the frozen fish 2. The improvement in the adhesion of the pad 63e to the surfaces of the frozen fish 2 improves the accuracy of the ultrasonic measurement. Therefore, when the pad 63e is cooled, becomes low temperature, and its property changes, the accuracy of the ultrasonic measurement may be reduced. Therefore, in the present embodiment, the temperature of the pad 63e is maintained by the heater 64. Further, the inclusion of the heater 64 makes it possible to melt the frost accumulated on the surface of the frozen fish 2. Then, the moisture resulting from the melting of the frost can be interposed between the pad 63e and the surfaces of the frozen fish 2. This water serves as a couplant. In the ultrasonic measurement, the couplant is typically supplied between the pad and a measurement target to enhance adhesion between the pad and the measurement target. According to the present embodiment, the ultrasonic measurement can be performed using water generated by melting frost as the couplant. In addition, the frost contains air, and the accumulation of the frost reduces the accuracy of the ultrasonic measurement. In the present embodiment, since the frost is melted by the heater, the accuracy of the ultrasonic measurement can be improved also in this respect. The frozen fish 2 of the present embodiment is edible, but in the present embodiment, the ultrasonic measurement can be performed without applying a couplant that cannot be applied to food. Although the heater 64 illustrated in FIGS. 13A to 13C is embedded in the pad holding plate 63c, the heater 64 may be provided so as to be exposed on the surface of the pad holding plate 63c. The heater 64 may be provided at a position other than the pad holding plate 63c.

Here, the temperature of the measurement head 63 will be described. The heater 64 operates so that the temperature of the measurement head 63 is 5 °C or more and 40 °C or less. The temperature of the measurement head 63 is set from the viewpoint of suppressing the curing of the material of the pad 63e, that is, silicon rubber in the present embodiment. The temperature of the measurement head 63 is preferably a temperature at which the frost accumulated on the surface of the frozen fish 2 can be melted. Theoretically, if the temperature of the measurement head 63 in contact with the frost is 0 °C or more, the frost can be melted. However, the temperature of the measurement head 63 decreases by contacting the frozen fish 2. Therefore, the temperature of the measurement head 63 is preferably 5 °C or more. When the temperature of the measurement head 63 is 5 °C or more, the ultrasonic measurement can be performed while suppressing the curing of the silicone rubber and melting the frost.

However, when the ultrasonic measurement is continuously performed on the plurality of frozen fish 2, the temperature of the measurement head 63 may decrease. Therefore, in order to continuously perform the measurement, the temperature of the measurement head 63 is set to 5 °C or more and 40 °C or less. This makes it possible to cope with continuous measurement.

Next, the force of the electric actuator 62 pressing the frozen fish 2 will be described. The electric actuators 62 preferably press the measurement head 63 against the frozen fish 2 with a force equal to or greater than 98 N. In the ultrasonic measurement, the higher the adhesion between the pad and the measurement target, the higher the measurement accuracy. The higher the force with which the measurement head 63 is pressed against the measurement target, the higher the adhesion between the pad and the measurement target. In the present embodiment, the lower limit of the force with which the electric actuators 62 press the measurement head 63 against the frozen fish 2 is set to 98 N, that is, about 10 kg weight. Even if the pressing force is lower than 98 N, the ultrasonic measurement can be performed.

On the other hand, the upper limit of the force with which the electric actuator 62 presses the measurement head 63 against the frozen fish 2 is not particularly limited. This is because the frozen fish 2 is very hard and it is difficult to assume that the frozen fish 2 is damaged, and when the pressing force is increased, the probe units are more likely to be damaged than the frozen fish 2.

The first probe unit 61a is mounted on the upper seventh coupling member 32g via a mounting bracket 621. The second probe unit 61b to the fourth probe unit 61d are also mounted on predetermined coupling members 32 in the same manner.

### [Configuration of Width Measurement Unit]

Next, the width measurement unit 70 will be described. The width measurement unit 70 corresponds to a second measurement unit. The width measurement unit 70 includes a sensor bracket 71 and a width measurement sensor 72. Referring to FIG. 8A, the width measurement unit 70 is provided on the second coupling member 32b. The width measurement sensor 72 is a camera using near infrared rays, and measures the width dimension of the frozen fish 2 conveyed by the conveyance unit 10. The width measurement sensor 72 is electrically connected to the support unit controller 82 that controls the drive unit 42 of the second support unit 40. The support unit controller 82 operates the drive unit 42 at the timing when the width measurement sensor 72 detects that the frozen fish 2 has been conveyed to the position of the predetermined width dimension. Thus, the frozen fish 2 can be fixed at the position of the width dimension determined each time. As a result, when the plurality of frozen fish 2 are measured, the ultrasonic measurement can be performed under the same conditions. That is, even when the length and weight of the frozen fish 2 are different, the ultrasonic measurement can be performed at the position of the fixed width dimension. As a result, the accuracy of the measurement of the quality of the frozen fish 2 is improved. The width measurement sensor 72 may be other than a sensor using near infrared rays.

### [Measurement Method]

Next, a method of measuring the frozen fish will be described.

### <Loading Step>

First, a loading step of the frozen fish 2 will be described. The frozen fish 2 is loaded into the conveyance unit 10 in the loading region 10a of the conveyance unit 10. The loading method of the frozen fish 2 is not particularly limited, but for example, the worker 1 moves the frozen fish 2 arranged on a placement table installed according to the height of the conveyance unit 10 to the loading region 10a of the conveyance unit 10 using a hook rod. A lifting device may be used, and the moving method is not limited.

The loaded frozen fish 2 is placed so that the longitudinal direction thereof coincides with the conveying direction and the head thereof is positioned on the upstream side in the conveying direction. At this time, the head thereof is brought into contact with the work pusher 21.

### <Conveying Step>

Next, the frozen fish 2 is conveyed toward the downstream side by operating the conveyance chain 19 of the conveyance unit 10. The frozen fish 2 is mounted on the conveyance chain 19, and conveyed toward the downstream side by pushing the head thereof by the work pusher 21.

### <Supporting Step>

The width of the frozen fish 2 conveyed toward the downstream side is measured by the width measurement unit 70 while the frozen fish 2 is moving. Then, at the timing when the width measurement sensor 72 detects that the frozen fish 2 has been conveyed to the position of the predetermined width dimension, a signal is transmitted to the support unit controller 82. The support unit controller 82 that has received the signal transmits a drive signal to the drive unit 42 to operate the drive unit 42. Thus, the first pressing unit 41a and the second pressing unit 41b are operated to fix and support the frozen fish 2 from the vertical direction and the width direction. This can suppress the movement of the frozen fish 2 and the change in the posture of the frozen fish 2.

### <Measurement Step>

In the measurement step, the measurement head 63 heated by the heater 64 is brought into contact with the frozen fish 2. Thus, the ultrasonic measurement is performed on the frozen fish 2 while melting the frost accumulated on the surfaces of the frozen fish 2 supported by the first pressing unit 41a and the second pressing unit 41b. The acquired measurement value is processed by the information processing unit 85 to determine the quality of the frozen fish 2.

### Other Embodiments

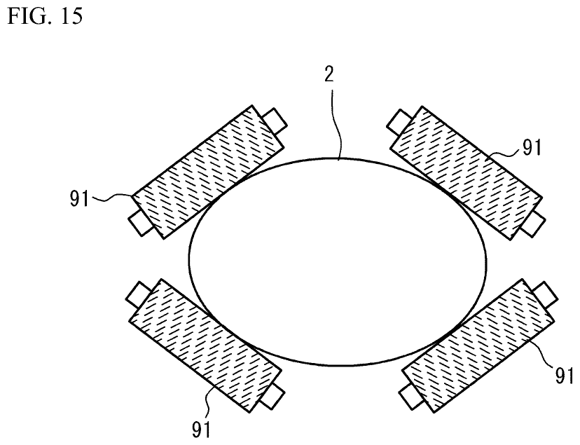

The ultrasonic measurement apparatus 100 may include a defrosting brush 91 as a removing unit that removes frost accumulated on the surface of the frozen fish 2 (FIG. 15). The ultrasonic measurement apparatus 100 includes the heater 64 that melts the frost. However, depending on the measurement environment of the frozen fish 2, it is assumed that the heater 64 alone is not enough to remove the frost in time. For example, when the ultrasonic measurement is performed in an environment at a room temperature of about 5 °C, the frost is not accumulated so much on the surface of the frozen fish 2 immediately after the frozen fish 2 is taken out from a freezer. However, a large amount of frost is accumulated on the surface of the frozen fish 2 taken out from the freezer after several minutes. In such a case, the frost is removed in advance by the defrosting brush 91, and then the measurement is performed by the ultrasonic measurement apparatus 100. Thus, the ultrasonic measurement can be performed by heating with the heater 64. Various modes of the defrosting brush 91 can be selected. The defrosting brush 91 illustrated in FIG. 15 has a brush formed around a rotating shaft, and removes the frost by rotating the rotating shaft. In this case, the rotation direction is not limited, and the rotor may be rotated in one direction or may be rotated alternately in both directions. The defrosting brush may also be in a circular motion. In this case, the rotation direction may be one direction, or may be a mode in which the rotation direction alternates between both directions. Further, instead of rotary motion, the linear reciprocating motion may also be used. That is, the movement of the defrosting brush is not limited.

### [Effects]

Hereinafter, the effects of the embodiments disclosed in the present specification will be described.

The ultrasonic measurement apparatus 100 of the present embodiment includes the work pusher 21 functioning as the first support unit, and the second support unit 40. This makes it possible to suppress the movement of the frozen fish 2 and suppress the change in the posture of the frozen fish 2 when the probe units 61a to 61d functioning as the first measurement unit are pressed against the frozen fish 2.

The ultrasonic measurement apparatus 100 according to the present embodiment includes the heater 64. Thus, the ultrasonic measurement can be performed while melting the frost on the surface of the frozen fish 2. At this time, since the water from which the frost is melted is used as the couplant, safety is high.

Further, since the temperature of the measurement head 63 is set to 5 °C or more and 40 °C or less by the heater 64, it is possible to suppress a decrease in the temperature of the measurement head 63 and to continuously measure the plurality of frozen fish 2.

The electric actuators 62 press the measurement head 63 against the frozen fish with the force equal to or greater than 98 N. This can increase the measurement accuracy.

By further providing the removing unit that removes the frost accumulated on the surface of the frozen fish 2, the ultrasonic measurement can be performed even when a large amount of frost is accumulated.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various change, substitutions, and alterations could be made hereto without departing from the spirit and scope of the invention.

What is claimed is:

1. An ultrasonic measurement apparatus comprising:
a conveyor that conveys a frozen fish from one side to another side;
a first measurer provided on the another side of the conveyor, the first measurer contacting the frozen fish conveyed by the conveyor to perform ultrasonic measurement on the frozen fish;
a support member that supports the frozen fish contacted by the first measurer; and
a heater provided in the first measurement, the heater increasing a surface temperature of the frozen fish.

2. The ultrasonic measurement apparatus according to claim 1, further comprising:
a second measurer that measure a dimension of the frozen fish conveyed by the conveyor in an intersecting direction that intersects a conveyance direction;
wherein the conveyor stops conveying the frozen fish based on a measurement result of the second measurer, and
the first measurer contacts the frozen fish that is stopped to perform the ultrasonic measurement.

3. The ultrasonic measurement apparatus according to claim 1, wherein
the support member comprises a first support member that suppresses movement of the frozen fish towards the one side of the conveyor due to contact of the first measurer with the frozen fish.

4. The ultrasonic measurement apparatus according to claim 3, wherein
the first support member comprises a brake device that sets the conveyor in a fixed state, and a stopper member that avoids the frozen fish from sliding on the conveyor fixed by the brake device.

5. The ultrasonic measurement apparatus according to claim 3, wherein
the support member comprises a second support member that is different from the first support member and that suppresses movement of the frozen fish due to contact of the first measurer with the frozen fish.

6. The ultrasonic measurement apparatus according to claim 5, wherein
the second support member comprises a first pressing member and a second pressing member that interpose the frozen fish from an intersecting direction that intersects a conveyance direction of the frozen fish.

7. The ultrasonic measurement apparatus according to claim 6, wherein
the first pressing member and the second pressing member include roller members provided with a gap therebetween, respectively.

8. The ultrasonic measurement apparatus according to claim 1, wherein
the heater heats the first measurer so that the temperature of the first measurer is 5 °C or more and 40 °C or less.

9. The ultrasonic measurement apparatus according to claim 1, wherein
the first measurer comprises a probe that comes into contact with the frozen fish and a pressing member that presses the probe against the frozen fish.

10. The ultrasonic measurement apparatus according to claim 9, wherein
the pressing member presses the probe against the frozen fish with a force equal to or greater than 98 N.

11. The ultrasonic measurement apparatus according to claim 1, further comprising:
a remover that removes frost accumulated on a surface of the frozen fish.

12. A method of measuring a frozen fish comprising:
conveying the frozen fish to a predetermined position from one side toward another side by a conveyor;
supporting the frozen fish conveyed to the predetermined position; and
bringing a probe heated by a heater into contact with the frozen fish, and melting frost accumulated on a surface of a supported frozen fish to perform ultrasonic measurement on the frozen fish.
